(19) United States
(12) Patent Application Publication
(10) Pub. No.: US 2025/0189442 A1
(43) Pub. Date: Jun. 12, 2025

(54) SCAVENGED ANESTHETIC SENSOR AND METHOD FOR SENSING SCAVENGED ANESTHETICS IN AN AIRFLOW

(71) Applicant: Blue-Zone Technologies Ltd., Concord (CA)

(72) Inventors: Ahmed NIRI, Concord (CA); Matthew Manuel WILLISTON, Concord (CA); Dusanka FILIPOVIC, Concord (CA); Eugene PALATNIK, Concord (CA)

(21) Appl. No.: 19/056,166

(22) Filed: Feb. 18, 2025

Related U.S. Application Data
(63) Continuation of application No. PCT/CA2023/051092, filed on Aug. 17, 2023.
(60) Provisional application No. 63/398,928, filed on Aug. 18, 2022.

(51) Int. Cl. G01N 21/3504; A61M 16/08; A61M 16/20; G01N 1/24

(52) U.S. Cl. CPC G01N 21/3504; G01N 1/24; A61M 16/085; A61M 16/202; A61M 2205/3313

(57) ABSTRACT

An anesthetic gas sensor including: a measurement cell; an inlet for receiving a sample to be tested; an outlet for releasing the sample; an infrared (IR) emitter; an IR detector to receive IR light from the IR emitter and determine an amount of IR light received through the measurement cell; and a processor to determine a concentration of anesthetic gas in the sample based on the amount of infrared light received versus the amount of infrared light received in a control sample. A method of sensing scavenged anesthetic gas in a scavenged gas flow, the method including: collecting a sample of the gas flow; passing infrared (IR) light through the sample; determining an amount of infrared light received through the sample; and determining a concentration of anesthetic in the sample. The gas sensor and method are intended to detect concentrations of 50 ppm of anesthetic gas or less.

### FIELD

The present disclosure relates generally to the sensing of scavenged anesthetics in an air flow. More particularly, the present disclosure relates to a scavenged anesthetic sensor and a method for sensing scavenged anesthetics in an air flow in order to control a bypass valve.

### BACKGROUND

Halogenated hydrocarbon compounds include the families of compounds: bromo-, fluoro- and/or chloro-ethers, fluorinated alkyl ethers, chlorofluorocarbons and chlorofluoro ethers and their derivatives. These families of compounds are typically used as solvents, refrigerants, anesthetic agents, aerosol propellants, blowing agents and the like. Many of these compounds are widely used and are routinely discharged into the atmosphere after their use.

Halogenated hydrocarbons used as anesthetic agents include the chemical formulae: 1,1,2-trifluoro-2-chloroethyl difluoromethyl ether and 1-chloro-2,2,2-trifluoroethyl difluoromethyl ether. These chemicals are also commonly known as “enflurane” and “isoflurane”, respectively. Other anesthetics include chemical formulae: 2,2,2-trifluoro-1-fluoroethyl-difluoromethyl ether and 2,2,2-trifluoro-1-[trifluoromethyl] ethyl fluoromethyl ether. These chemicals are commonly known as “desflurane” and “sevoflurane”, respectively.

These anesthetics are highly volatile organic compounds, produced in a liquid form and, in use, are evaporated using an anesthetic specific vaporizer to be mixed with carrier medical gases, such as nitrous oxide, oxygen and/or medical air to a low concentration dosage at a low flow before being administered to a patient as an inhalation anesthetic in surgical operations conducted under general anesthesia, in sedation, or the like. These volatile anesthetic agents are effective in small dosages. Typical dosages in the carrier gases range between 0% to 8.5%, depending on the conditions evaluated and monitored by the anesthetist. Less than 5% of inhalational anesthetic administered to the patient is metabolized by the patient, resulting in the majority of administered anesthetics to be exhaled in the patient breathing circuit. The leftover anesthetics are redirected with other gases, such as CO2, from the anesthetic gas machine, typically into a scavenging system to be released into the atmosphere. The scavenged gas stream exiting the anesthetic gas machine includes scavenged anesthetics and contains entrained CO2, moisture and possibly some by-products that generally result from the patient exhale and anesthetic gas mix recirculation stream. For safety reasons, hospitals typically employ vacuum pump(s) in their Anesthetic Gas Scavenging System (AGSS) to ensure all scavenged anesthetic is evacuated from both the anesthetic gas machine outlet (avoid potential build-up in patient breathing circuit) and the operating rooms (avoid potential build-up in room air from leaks in breathing masks, etc.). This AGSS involves drawing in room air, past the anesthetic gas machines scavenging block, which is then mixed with the scavenged gas from the anesthetic gas machine, resulting in increased flow and anesthetic dilution in the gas mix through the hospital's AGSS that is subsequently discharged to the atmosphere. Importantly, addition of room air (@ Dew Point) or the like in the AGSS piping and the anesthetic gas machines in operating rooms is typically continuous regardless of whether the operating room is in use or not, i.e., regardless of anesthetic presence in the scavenged gas. The addition of room air or the like is used to purge any scavenged anesthetics from the anesthetic gas machines and to maintain an air flow to keep any scavenged anesthetics in the system moving away from the anesthetic gas machines and away from the operating rooms.

Halogenated hydrocarbon compounds, and, in particular, inhalation anesthetics, are greenhouse gases and can cause environmental issues if released into the atmosphere. Systems are available by which scavenged inhalation anesthetics can be removed from the scavenged gas before the scavenged gas is discharged into the atmosphere, however, these systems may fail, may become overloaded, or the like. Further, these systems can sometimes function more efficiently when only air flows containing scavenged inhalation anesthetics are passed through the system. For example, to reduce co-adsorption of water, prevent other impacts on the adsorbents or the like, it is desirable to bypass the scavenged anesthetic removal system when the operating room is not in use of inhalation anesthetics. In these cases, it is helpful to have a sensor that can detect presence of scavenged anesthetic in an AGSS airflow and control the bypass valve actuator such that the air flow can be directed to the system for scavenged inhalation anesthetic removal when present or directed to bypass the removal system when anesthetics are not present. In some cases, the air flow can be directed to a secondary system or the like if there is a breakthrough of scavenged anesthetics from the removal system itself. While some sensors are available for sensing halogenated hydrocarbons, they have generally been designed for use in different applications, such as sensing at anesthetic gas machines for monitoring of anesthetic concentrations delivered to the patient, for leaks, and the like. These sensors do not need to be as sensitive because of the relatively high concentrations in these environments and are also not configured to control a bypass valve actuator.

It is, therefore, desirable to provide an improved sensor and method for sensing scavenged inhalation anesthetics.

The above information is presented as background information only to assist with an understanding of the present disclosure. No determination has been made, and no assertion is made, as to whether any of the above might be applicable as prior art with regard to the present disclosure.

### SUMMARY

In a first aspect, there is provided an anesthetic gas sensor including: a measurement cell; an inlet to the measurement cell for receiving a sample to be tested; an outlet from the measurement cell for releasing the sample; an infrared (IR) emitter provided to the measurement cell; an IR detector provided to receive IR light from the IR emitter and configured to determine an amount of IR light received through the measurement cell; and a processor to determine a concentration of anesthetic gas in a test sample based on the amount of infrared light received versus the amount of infrared light received in a control sample.

In some cases, the measurement cell may be an elongated cylinder, the IR emitter may be at one end of the cylinder, and the IR detector may be at the other end of the cylinder.

In some cases, the IR emitter, IR detector, and processor may be configured to detect concentrations of less than 20 ppm, 15 ppm, 10 ppm, 5 ppm of anesthetic gas or the like.

In some cases, the anesthetic gas sensor may further include a filter to control the frequency of the IR light between the IR emitter and IR detector.

In some cases, the anesthetic gas sensor may further include an output/display connected with the processor to output a status of the sensor.

In some cases, the anesthetic gas sensor may further include an output/display connected with the processor to output a result obtained by the sensor.

In some cases, the anesthetic gas sensor may further include a relay controlled by the processor to activate a valve based on a result obtained by the sensor.

In some cases, the processor may control the inlet to allow the sample to enter the measurement cell, operate the IR emitter and IR detector to determine an amount of infrared light received through the sample, and determine a concentration of anesthetic in the sample based on the amount of infrared light received versus the amount of infrared light received in a control sample.

In some cases, the anesthetic gas sensor may further include a pump to bring the sample into the measurement cell.

According to another aspect herein, there is provided a method of sensing scavenged anesthetic gas in a scavenged gas flow, the method including: collecting a sample of the gas flow; passing infrared (IR) light through the sample; determining an amount of infrared light received through the sample; and determining a concentration of anesthetic in the sample based on the amount of infrared light received versus the amount of infrared light received in a control sample.

In some cases, the method may further include activating a bypass valve actuator when the concentration of anesthetic may be below a predetermined threshold such that an anesthetic gas collection system may be bypassed. In this case, the predetermined threshold may be 50 ppm, 40 ppm, 30 ppm, 20 ppm, 10 ppm, 5 ppm of anesthetic gas or the like.

In some cases, the method may further include activating an alert when the concentration of anesthetic may be above a predetermined threshold. In this case, the predetermined threshold may be 50 ppm, 40 ppm, 30 ppm, 20 ppm, 10 ppm, 5 ppm of anesthetic gas or the like.

In some cases, the method may further include: collecting the control sample from ambient air; passing infrared (IR) light through the control sample; and determining an amount of infrared light received through the control sample.

Other aspects and features of the present disclosure will become apparent to those ordinarily skilled in the art upon review of the following description of specific embodiments in conjunction with the accompanying figures.

### BRIEF DESCRIPTION OF FIGURES

Embodiments of the present disclosure will now be described, by way of example only, with reference to the attached Figures.

FIG. 1A illustrates a schematic of a central scavenged anesthetic gas collection system for capturing scavenged anesthetics from multiple anesthetic gas machines;

FIG. 1B illustrates a schematic of another central scavenged anesthetic gas collection system for capturing scavenged anesthetics from multiple anesthetic gas machines;

FIG. 2 is a schematic cross-sectional view of an embodiment of a gas cell in a sensor for anesthetics;

FIG. 3 is a view of an embodiment of a sensor for scavenged anesthetics;

FIG. 4 is a schematic wiring diagram for the sensor of FIG. 3;

FIG. 5 is a flowchart of an embodiment of a method for sensing scavenged anesthetics.

### DETAILED DESCRIPTION

In the following, various example systems and methods will be described herein to provide example embodiment(s). It will be understood that no embodiment described below is intended to limit any claimed invention. The claims are not limited to systems, apparatuses or methods having all of the features of any one embodiment or to features common to multiple or all of the embodiments described herein. A claim may include features taken from any embodiment as would be understood by one of skill in the art. The applicants, inventors or owners reserve all rights that they may have in any invention disclosed herein, for example the right to claim such an invention in a continuing or divisional application and do not intend to abandon, disclaim or dedicate to the public any such invention by its disclosure in this document.

Generally, the present application relates to a halogenated hydrogen sensor that can be used with a system for capturing a variety of halogenated hydrocarbons from a gas stream. Compounds generally known as halogenated hydrocarbons include bromo-, chloro-and/or fluoro-ethers, fluorinated alkyl ethers, chloro-fluorohydrocarbons, chlorofluoroethers and their derivatives. Inhalation anesthetics are well known types of halogenated hydrocarbons which include isoflurane, enflurane, halothane, methoxyflurane, desflurane, and sevoflurane.

Other halogenated hydrocarbons include the variety of refrigerant gases, such as Freons™ (which include trichlorofluromethane, and dichlorodifluoromethane). This family of halogenated hydrocarbon compounds includes, for example, an alkyl group or ether group substituted with one or more of chloro-, fluoro-and bromo-groups.

While the following description relates to the sensing of various inhalation halogenated anesthetics, it is appreciated that the principles of the application, which are demonstrated by the embodiments, can be applicable to the recovery of other types of halogenated hydrocarbons.

During surgery, a patient can be treated with inhalation anesthetics. It is appreciated that the use of the term “patient” is in a general sense and should not be limited to human patients. It is understood that anesthesia is practiced on a variety of mammals, not only humans, but also animals such as horses, cattle and other forms of livestock, domestic pets and the like.

The inhalation anesthetics are typically delivered in combination with “medical air” as a carrier gas, which is typically a combination of oxygen and/or nitrous oxide and/or medical air. A slip stream of the exhaled gas mix is recirculated back to the patient after passing through a CO2 absorber. As the patient breathes the gas stream containing the anesthetic (with support of a respirator), a desired degree of unconsciousness is achieved and monitored by the anesthetist. Typically, a very small portion of the anesthetic is adsorbed or metabolized by the patient, less than 5% depending on which anesthetic agent is used. Flow rates of the gas stream to the patient, in the patient breathing circuit, may be in the range of 0.2 to 7 liters per minute, where the concentration by volume of the halogenated anesthetic fluctuates during the operation and may be in the range of 0% to 8.5% depending on numerous factors and conditions evaluated and monitored by the anesthetist.

It is important to ensure that the gas mixture containing anesthetic being administered by the anesthetic gas machine is not exhausted into the operating theatre, because exposure to the anesthetics is a recognized health and occupational hazard that can have both short term and long-term effects on the staff in the operating room. As such, scavenging systems (sometimes called anesthetic gas scavenging systems, “AGSS”) are typically provided to carry the flow of the anesthetic gases from the patient exhalent outside of the operating room.

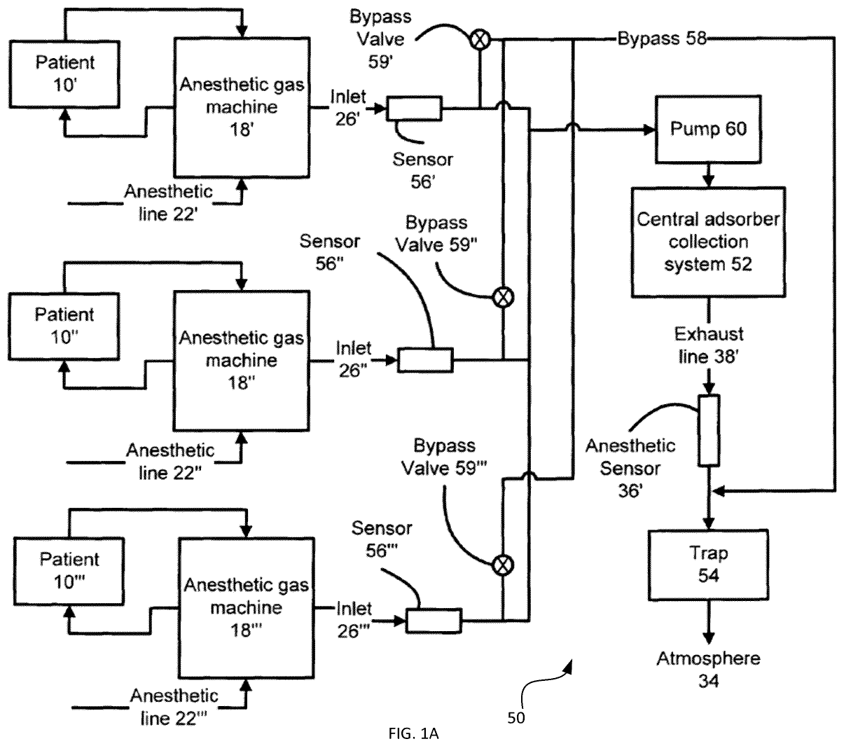

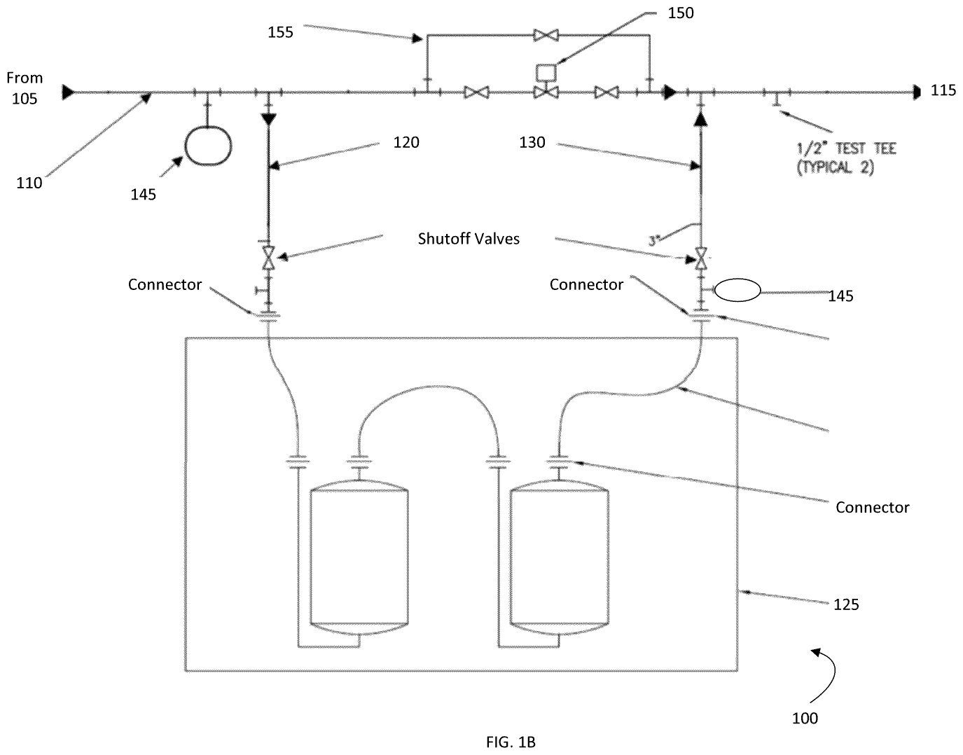

FIG. 1A illustrates an embodiment of a central collection system 50 in which anesthetics from more than one operating room are captured by a central adsorber collection system 52. The central collection system 50 gathers anesthetics from multiple anesthetic gas machines (in FIG. 1A, 18′, 18″ and 18″), which may be in multiple operating rooms via a gas scavenging system or the like. The gas scavenging system connects to the central collection system via inlet lines 26′, 26″ and 26″. The central collection system 50 includes the central adsorber collection system 52, which includes one or more canisters (detailed below) charged with at least one adsorbent which selectively adsorbs the anesthetics from the combined gas streams from the anesthetic gas machines. The central collection system 50 is intended to leave the discharge stream substantially free of anesthetics for ultimately exhausting to atmosphere 34. As shown in FIG. 1A, the air flow can be driven by a pump 60 that may be part of the central collection system 50 (as generally shown). In some cases, if there is a pump 60 as a part of the central collection system 50, there may need to be another pump (not shown) on the bypass line 58. In some further cases, a pump may be a part of a general anesthetic/medical gas scavenging system (sometimes called AGSS), which gathers air/gases from various areas of a hospital or operating rooms to send the gas to the central collection system 50 or the central adsorber collection system 52. The pump may be arranged to pull or push air flow depending on where it is located.

In some embodiments of the central collection system 50, sensors 56′, 56″ and 56″ may be provided to inlet lines 26′, 26″ and 26″ and configured to sense the presence of anesthetics in the medical air flowing through the inlet lines. In cases where no anesthetics are detected, inlet lines 26′, 26″ and/or 26″ may be closed off to isolate the appropriate operating room from the central collection system 50. The inlet lines 26′, 26″ and 26″ may alternatively be routed, via bypass 58, so that the medical air (without anesthetic or with a low amount of anesthetic) is sent to the atmosphere and away from the central collection system 50. Closing off the inlet lines 26′, 26″ and 26″ or routing the medical air through bypass 58 can be achieved using bypass valves 59′, 59″ and 59′″.

The use of a bypass 58 can be helpful because, for example, there may be cases where the medical air supply is left on even though no anesthetic is being used. In such a situation, it is undesirable to continue to flow medical air through the central collection system 50 since the continuous flow of medical air can slowly desorb anesthetics previously captured and moisture from the medical air flow may be adsorbed limiting capacity for future capture of anesthetic.

An anesthetic sensor 36′ may be provided in the central collection system exhaust line 38′ to sense the presence of anesthetics exiting the central adsorber collection canister. The anesthetic sensor 36′ would sense when anesthetics have broken through the adsorbent into the exhaust and prompt a user with feedback (e.g. an alarm) to change the adsorbent by, for instance, replacing one or more adsorbers, replacing the adsorbent in one or more adsorbers, or regenerating the adsorbent. It is appreciated that an adsorption front of adsorbed anesthetics travels along the bed of adsorbent in a canister towards the outlet as the anesthetic is adsorbed. Such an adsorption front will usually have a curved profile across the canister as it approaches the outlet. The sensor 36′ is intended to sense when any portion of the adsorption front has broken through the adsorbent into the outlet. The anesthetic sensor 36′ may be connected to a remote monitoring station (not shown) or the like. The remote monitoring station may be equipped with an alarm which is actuated when the anesthetic sensor 36′ senses anesthetics in exhaust line 38′ and indicate to a technician that a canister should be replaced so that continued recovery of anesthetics is achieved.

In some embodiments, a trap 54 may be provided in the exhaust line 38′ or on the bypass line 58 or on both, located before the system exhausts to the atmosphere 34, in order to reduce the possibility that anesthetics are released to the atmosphere. The trap 54 may have the same adsorbent as that in the central adsorber collector canister, or may have a different adsorbent, such as activated carbon, silicalite, molecular sieve, or the like. In some cases, the bypass line 58 may not have a trap 54 and vent directly to atmosphere.

An alternative to providing the anesthetic sensor 36′ for sensing an anesthetic in the exhaust line is to provide a weight sensor (e.g. a load cell) such that the weight of the capture device (or adsorbent or canisters) could be monitored and the user could be prompted to change or regenerate the adsorbent once a predetermined weight of anesthetic, based on known adsorbent collection capacity, was collected in the central adsorber collection system. This weight sensor could also be provided in addition to the anesthetic sensor 36′ for additional protection or confirmation.

In another embodiment, historical loading patterns could be used to determine an appropriate time to change or regenerate the adsorbent or to determine an appropriate adsorber size to support a given period of collection. Replacement or regeneration of the adsorbent in any of the above alternatives may be desirable even though the adsorbent is not fully saturated with anesthetic.

One of the challenges involved in a central collection system 50 relates to the flow rate and concentration of anesthetics in the flow. Typically, a hospital scavenging system uses the pump 60 or some form of air moving device located before the central collection system to draw the gas stream from various operating rooms into the scavenging system. This device draws the gas stream originating from the anesthetic gas machines 18′, 18″ and 18″, as well as additional “make up” air. An increased flow rate due to the makeup air results in a dilution of the concentration of anesthetics. This dilution can be, for example, in the range of 1:20 (volume of anesthetic gas stream from the anesthetic gas machine: volume of flow entering the hospital scavenging system and passing through the central collection system) since the flow rate from an anesthetic gas machine can be about 2 L/min, while the flow rate entering the hospital scavenging system can be 40 L/min. Given the dilution of anesthetics passing through the central collection system, the central adsorber collection system 52 and/or the central collection system 50 more generally should be designed to ensure that the residence time is adequate for the adsorbent material to adsorb the anesthetics. Changing the residence time can be done by changing the volume of the central adsorber collection system 52, by changing the flow rate of the gas, by incorporating multiple canisters in series or in parallel, or the like. It is appreciated that the relationship between these variables is given by the equation: Residence Time=Capture Device Volume/Gas Flow Rate.

FIG. 1B shows another embodiment of a central collection system 100 according to an embodiment herein. In this embodiment, there is an anesthetic gas scavenging system (AGSS) 105 in place, which gathers air flow from a plurality of operating rooms and transfers the air flow into piping 110 for transmission to a location, typically near a roof of the hospital, where it can be vented 115. In a typical case, there will be a fan/pump in the system to move air through the AGSS. In this case, the central collection system 100 is connected with the AGSS 105. The central collection system 100 includes an inlet line 120 that is connected to a central adsorber collection system 125, which may include one or more canisters (two canisters shown) charged with at least one adsorbent which selectively adsorbs the anesthetics from the AGSS gas stream. The central adsorber collection system 125 is then connected back to the piping 110 of the AGSS 105 closer to the vent 115 by an outlet line 130. The central collection system 100 may include a gas sensor 145 provided prior to the inlet line 120 for sensing the presence of anesthetic gas in the air flow in the piping 110. If anesthetic gas is sensed, an electronic control valve 150 provided to the piping 110 can be closed such that the air flow will be directed to inlet line 120 and the central adsorber collection system 125. In some cases, the sensor may also trigger a control valve on the input line to allow gas flow into the containers, however, this may not be needed as the air flow will generally follow the more direct path to the vent. If anesthetic gas is not sensed, an electronic control valve 150 provided to the piping 110 can be opened such that the air flow will be directed to bypass the central adsorber collection system 125 by flowing directly from piping 110 to vent 115. There may also be a further anesthetic gas sensor on the outlet line 130 to sense if any anesthetic is “breaking through” the adsorbent in the canisters. In this case, an alarm could be triggered, and the flow could be redirected or the like.

The piping 110 may also be provided with a safety bypass line 155 that passes around the control valve 150 in case of some failure of the control valve 150 and/or the central adsorber collection system 125. In this case, the safety bypass may include an overpressure valve or the like, which will only activate if there is too much pressure on the safety bypass line.

The central adsorber collection system 125 may also include attachment connectors and shutoff valves on each of the inlet line 120 and the outlet line 130 so that the central adsorber collection system 125 can be safely removed from the system or exchanged. In this case, the air flow can be directed from piping 110 through the safety bypass line 155 and to the vent 115 regardless of the position of the control valve 150. The canisters may also have canister attachment connectors so that individual canisters can be removed from the central adsorber collection system 125.

In the embodiment shown in FIG. 1B, there are two canisters. It will be understood that the connection between the two canisters may also include a sensor and electronic bypass valve such that, if there is no anesthetic in the air flow after the first canister, the air may proceed directly to the vent and does not go through the second canister. It will also be understood that the canisters may serve different purposes, for example, one may be used as “a dryer” to first capture water from the air flow and the second canister may include an adsorbent to capture halogenated anesthetics from the dry air flow. Alternatively, as explained below, the canisters may be arranged in series or parallel and in various numbers and combinations depending on the needs of the overall system.

With these concepts in mind, the following description relates to embodiments of an anesthetic gas sensor 56, 145 and a method of sensing in further detail.

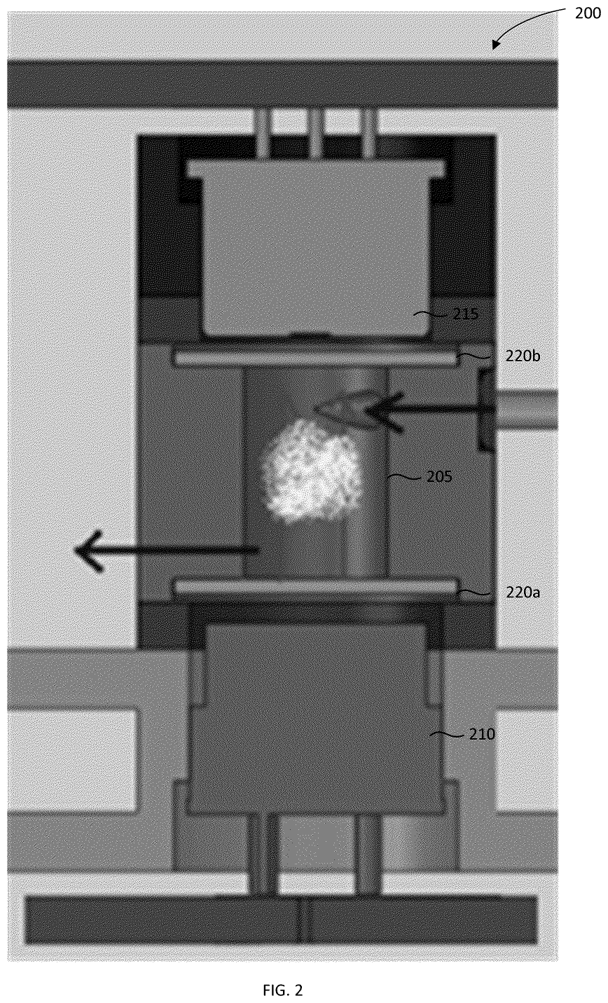

FIG. 2 is a schematic cross-section view of an embodiment of a gas cell of an anesthetic gas sensor 200. The sensor 200 is based on non-dispersive infrared (IR) spectroscopy and is intended to measure IR light absorption by anesthetic molecules in a gas sample contained in a measurement cell. As shown in FIG. 2, a cylindrical measurement cell 205 is placed between an infrared (IR) emitter 210 and an IR detector 215. In some cases, the measurement cell 205 may be cylindrical and made of stainless steel and the IR emitter may be at a base of the measurement cell 205 and the IR detector 215 at a top of the measurement cell 205. The measurement cell 205 may be separated from the IR emitter 210 and IR detector 215 by transparent covers/windows 220a, 220b such as glass or the like. A gas sample (cloud) is delivered into the measurement cell 205 (upper horizonal arrow) and the IR emitter radiates an IR beam, which may be, for example, a broad band far IR beam, which passes from the bottom window 220a through the gas sample, and reaches the IR detector through the top window 220b. As the IR beam travels through the measurement cell 205 (vertical arrows), the IR beam will be at least partly absorbed by anesthetic gas molecules in the sample, if any. After testing, the gas sample can be released from the measurement cell (lower horizontal arrow).

In some embodiments, the windows 220a, 220b or one of them, may include a filter or the like to adjust the wavelength of IR light used in order to target the presence of a particular anesthetic gas based on differences in the absorbtion of IR light by the anesthetic gas. In some cases, the window(s) may be automatically changed depending on the sensing to be conducted. It may be useful to test for specific anesthetics to determine the types of anesthetics in a particular canister. This can facilitate later processing if the types of anesthetics are known. In this embodiment, the IR emitter and detector are across from each other, but this configuration can be changed depending on the needs of the sensor. In some cases, the sensor may use fiber optics, mirrors, or the like to provide other configurations.

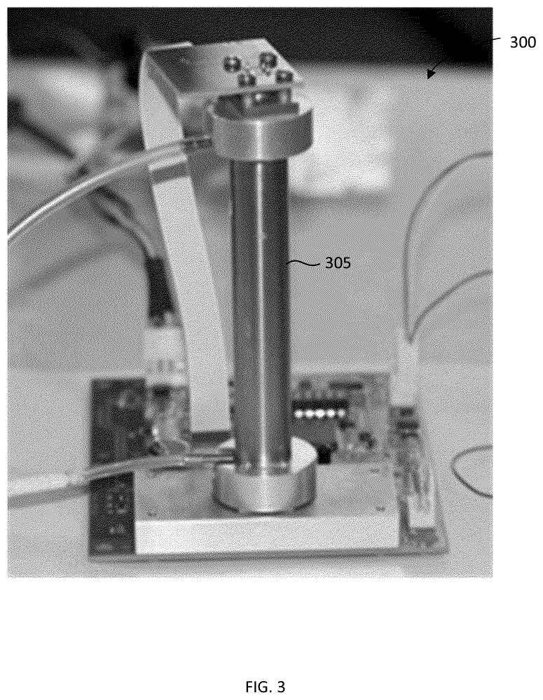

FIG. 3 shows another embodiment of an anesthetic gas sensor 300. As shown in FIG. 3, a measurement cell 305 can be an elongated cylinder or the like to be able to handle a low anesthetic gas concentration and facilitate detection in the low ppm range. In some cases, the measurement cell 205 or 305 can be a different shape than cylindrical. To determine the detection range of the sensor 300 and calibrate, the sensor 300 can be run using samples with known concentrations. In some embodiments, a gas sample measurement from the sensor can automatically be compared to an ambient air intake measurement. The difference between the gas sample measurement and the ambient measurement (sometimes referred to as the “delta”) is proportional to the anesthetic gas concentration in the gas sample and is used for sensing.

In some embodiments, the anesthetic gas sensor has an output or display to show the status of operation or the like. For example, the sensor may include LED lights such as: Green for “Measure”; Yellow for “Pause” and Red for “Detect”. In other embodiments, the anesthetic gas sensor may include a digital display showing the most recent sample result and include controls to adjust the delta threshold/value required to actuate the bypass valve.

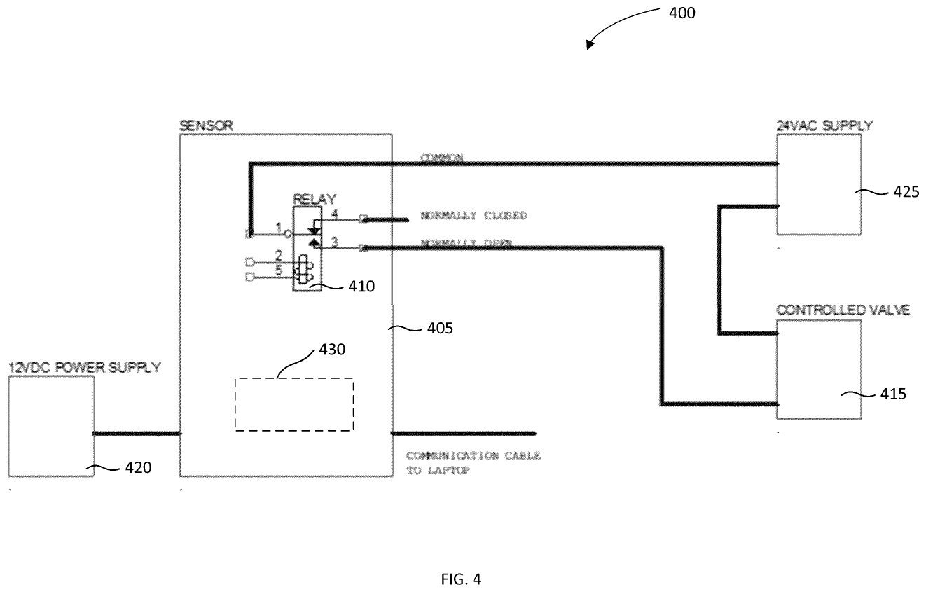

FIG. 4 is an embodiment of a circuit 400 including an anesthetic gas sensor 405, which may be similar to those shown in FIG. 2 or 3. In this embodiment, the anesthetic gas sensor includes a relay 410 that is configured to operate a valve 415. The anesthetic gas sensor 405 is powered by a power supply 420, in this case a 12 VDC power supply, and the valve 415 is powered by a second power supply 425, in this case a 24 VAC power supply. In this example, the relay may include three contacts: common, normally closed and normally open. In a prototype anesthetic gas sensor, common and normally open were used to control the valve via the second power supply. It will be understood that a different combination of relay contacts can be configured, depending on the valve 415 used. In some cases, the sensor 405 and/or the relay 410 may include a processor/microcontroller 430 and/or be connected to a processor, via a communication port/cable or the like.

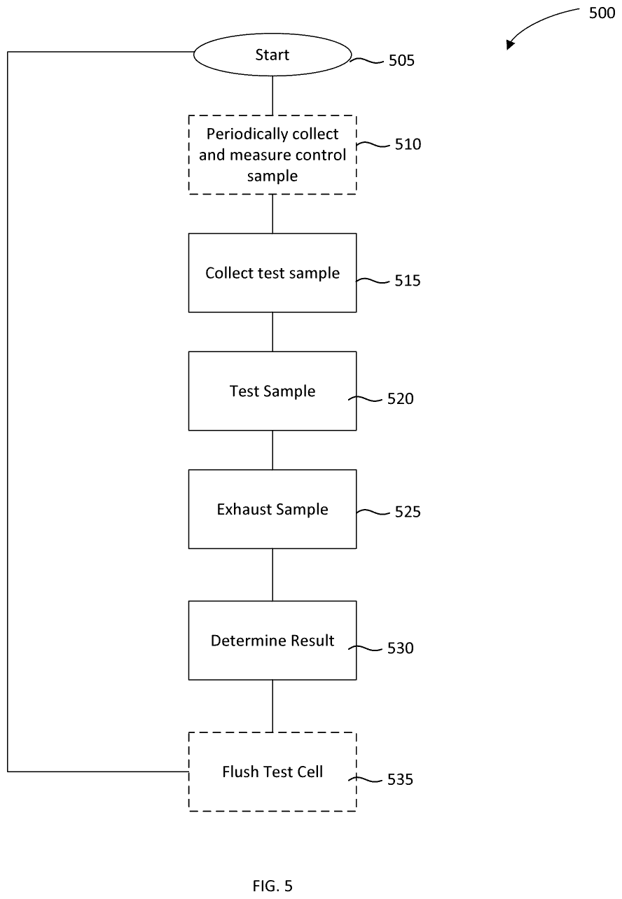

In operation, the microcontroller 430 can be programmed in various ways. In one example, the sensor is set to automatically operate in cycles and the cycles can be set for a predetermined period of time. In this case, illustrated in a flowchart showing an embodiment of a method 500 in FIG. 5, a programmed sequence may include a start at 505, for example, a Green LED “Measure” light turns on. At this point, ambient air can be collected (a control sample), and a base line (or control) reading is established at 510. Next, in this embodiment, about 80 ml of the air flow is collected in the measurement cell at a flow rate of approximately 160 ml/min for 30 seconds to obtain a sample at 515. It will be understood that the sample size and flow rate may vary depending on various factors such as the overall flow rate, the timing of the testing, the measurement cell size, the concentration of anesthetics (expected or otherwise) in the flow, and the like. Sample size may be in the range of, for example, 60-100 ml, 50-110 ml, or an appropriate range. In some cases, the gas sensor will include a pump for collecting the sample. After the sample is collected, a test reading is established at 520. The sample is then evacuated at 525 through an exhaust port. It is noted that the amount of anesthetic gas in a test sample will be negligible and would not pose a health risk when evacuated into the local environment. Based on the highest anesthetic gas concentration found in gas samples taken from existing AGSS gas flows, i.e., 1530 ppm, each test sample would contain up to 0.001 g of anesthetic gas. If operated for 5 hours, then a total of 100 samples taken by the sensor would lead to only 0.1 g anesthetic gas exhausted into the room. A standard HVAC system would generally prevent any build-up from occurring, such that anesthetic gas concentrations in the room will be below National Institute of Occupational Safety and Health (NIOSH) exposure limits.

The sensor/microcontroller then calculates the difference/delta between the baseline and the test sample to determine a result at 530. The delta is proportional to anesthetic presence in the test sample and controls the by-pass valve to open or close as described herein. As such, the delta can provide a test result indicating the parts-per-million (ppm) of anesthetic gas or the like.

Once the delta is calculated (and a result is obtained), the Green LED light turns off and the Yellow “Pause” LED light turns on. The system pauses for a predetermined/configurable period (such as, for example, 10, 20, 30, 40, 50 seconds, 1, 2, 3 minutes or the like). During the pause, the measurement cell may be flushed with ambient air at 535. The method then returns to the start (i.e., the Green “Measure” LED step for the next measurement cycle). In this way, a full cycle delta calculation can occur about every few minutes or as otherwise configured.

In the above example, a baseline or control sample of similar volume is collected during each test in order to provide as accurate a result as possible. In some embodiments, the baseline or control sample may only be taken periodically, for example, once per ten minutes, twenty minutes, thirty minutes, one hour, half day, whole day, week, or the like. Further, in some embodiments, the sensor may be configured to compare control samples from different times to each other to determine differences in the ambient air from test to test. If there is a change in the ambient air outside of a predetermined threshold, the controller may issue an alarm or the like. In this way, it may be possible to detect a leak of anesthetic gas or the like.

At 530, if the test result exceeds a predetermined threshold such as 5, 10, 20, 50 ppm or some number in these ranges, the red “Detect” LED can turn on and the by-pass valve can be triggered to close (or remain closed) directing the gas flow through the central collection system. If a test result is below the predetermined threshold, the red “Detect” LED light does not turn on and the by-pass valve is triggered to open (or remain opened) and the gas flow by-passes the central collection system. It will be understood that the turning on/off of the valve may also be controlled in an opposite way.

Embodiments of the anesthetic gas sensor are intended to sense concentrations as low as 0 to 2000 ppm, 0-1000 ppm, 0-500 ppm, 0-200 ppm or the like. Embodiments have been tested at detection levels as low as 15 ppm, 10 ppm and 5 ppm.

In the above embodiments, the flow rates in the AGSS are at a higher rate, such that taking samples from the air flow is more accurate for testing. If the flow rate in the AGSS is slower or other conditions allow, it may be possible to sample/test the air flow in the tube itself by configuring the sensor and/or IR emitter/detector appropriately in relation to the air flow.

As noted above, the sensor can also be used to sense when there is anesthetic gas in the air flow beyond the canister and to provide an alert when an anesthetic capture device is full or the like. This safety feature can be effective for devices that administer inhalation anesthetics in rooms that may not be connected to an AGSS system or the like. In particular, anesthetics are sometimes used in ICUs and MRI rooms, where a device using the anesthetic may not be vented to an AGSS or to outside air or the like. In this setting, an embodiment of a sensor of the type described herein could monitor an outlet of a capture device and alert that the capture device should be changed promptly so that anesthetics do not exhaust into the room, exposing hospital staff.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding. For example, specific details are not provided as to whether the embodiments or elements thereof described herein are implemented as a software routine, hardware circuit, firmware, computer program instructions on a computer readable medium, or a combination thereof.

The above-described embodiments are intended to be examples only. Alterations, modifications and variations can be effected to the particular embodiments by those of skill in the art without departing from the scope, which is defined by the claims appended hereto.

What is claimed is:

1. An anesthetic gas sensor comprising:
a measurement cell;
an inlet to the measurement cell for receiving a sample to be tested;
an outlet from the measurement cell for releasing the sample;
an infrared (IR) emitter provided to the measurement cell;
an IR detector provided to receive IR light from the IR emitter and configured to determine an amount of IR light received through the measurement cell; and
a processor to determine a concentration of anesthetic gas in a test sample based on the amount of infrared light received versus the amount of infrared light received in a control sample.

2. An anesthetic gas sensor according to claim 1, wherein the measurement cell is an elongated cylinder, the IR emitter is at one end of the cylinder, and the IR detector is at the other end of the cylinder.

3. An anesthetic gas sensor according to claim 1, wherein the IR emitter, IR detector, and processor are configured to detect concentrations of less than 15 ppm of anesthetic gas.

4. An anesthetic gas sensor according to claim 1, further comprising a filter to control the frequency of the IR light between the IR emitter and IR detector.

5. An anesthetic gas sensor according to claim 1, further comprising an output connected with the processor to output a status of the sensor.

6. An anesthetic gas sensor according to claim 1, further comprising an output connected with the processor to output a result obtained by the sensor.

7. An anesthetic gas sensor according to claim 1, further comprising a relay controlled by the processor to activate a valve based on a result obtained by the sensor.

8. An anesthetic gas sensor according to claim 1, wherein the processor controls the inlet to allow the sample to enter the measurement cell, operates the IR emitter and IR detector to determine an amount of infrared light received through the sample, and determines a concentration of anesthetic in the sample based on the amount of infrared light received versus the amount of infrared light received in a control sample.

9. An anesthetic gas sensor according to claim 1, further comprising a pump to bring the sample into the measurement cell.

10. A method of sensing scavenged anesthetic gas in a scavenged gas flow, the method comprising:
collecting a sample of the gas flow;
passing infrared (IR) light through the sample;
determining an amount of infrared light received through the sample; and
determining a concentration of anesthetic in the sample based on the amount of infrared light received versus the amount of infrared light received in a control sample.

11. A method according to claim 10, further comprising activating a bypass valve actuator when the concentration of anesthetic is below a predetermined threshold such that an anesthetic gas collection system is bypassed.

12. A method according to claim 11, wherein the predetermined threshold is 50 ppm of anesthetic gas.

13. A method according to claim 10, further comprising activating an alert when the concentration of anesthetic is above a predetermined threshold.

14. A method according to claim 13, wherein the predetermined threshold is 50 ppm of anesthetic gas.

15. A method according to claim 8, further comprising:
collecting the control sample from ambient air;
passing infrared (IR) light through the control sample; and
determining an amount of infrared light received through the control sample.
